(19) United States
(12) Patent Application Publication
(10) Pub. No.: US 2023/0233077 A1
(43) Pub. Date: Jul. 27, 2023

(54) METHODS AND RELATED ASPECTS FOR OCULAR PATHOLOGY DETECTION

(71) Applicant: THE JOHNS HOPKINS UNIVERSITY, Baltimore, MD (US)

(72) Inventors: Tin Yan Alvin LIU, Baltimore, MD (US); Zelia M. CORREA, Miami, FL (US)

(73) Assignee: THE JOHNS HOPKINS UNIVERSITY, Baltimore, MD (US)

(21) Appl. No.: 18/001,992

(22) PCT Filed: Jun. 29, 2021
(86) PCT No.: PCT/US2021/039612

§ 371 Date: Dec. 15, 2022

Related U.S. Application Data
(60) Provisional application No. 63/045,747, filed on Jun. 29, 2020.

(51) Int. Cl. A61B 3/14; G06T 7/00; G16H 50/20; G16H 50/70; G16H 10/60

(52) U.S. Cl. CPC A61B 3/14; G06T 7/0012; G16H 50/20; G16H 50/70; G16H 10/60; G06T 2207/20081; G06T 2207/30041; G06T 2207/20084

(57) ABSTRACT

Provided herein are methods of detecting a ophthalmologic genetic disease in a subject that include matching properties of captured images and/or videos with properties of an ocular pathology model that is trained on a plurality of reference images and/or videos of ocular cells of reference subjects, which properties of the ocular pathology model are indicative of the pathology. Related systems and computer program products are also provided.

### CROSS-REFERENCE TO RELATED APPLICATIONS

This application claims priority to U.S. Provisional Patent Application Ser. No. 63/045,747, filed Jun. 29, 2020, the disclosure of which is incorporated herein by reference.

### BACKGROUND

In recent years, artificial intelligence (AI) in the form deep learning (DL) has generated immense interest in the medical field. Briefly, DL methods are representation learning methods that use multi-layered neural networks, the performance of which can be enhanced using backpropagation algorithms to change reiteratively the internal parameters.¹ DL can be used to classify medical images accurately, and it has been applied in a wide variety of medical disciplines, especially in specialties where large, well-annotated datasets are more readily available, such as pathology,²⁻⁴ radiology,⁵⁻⁸ ophthalmology and oncology. Within ophthalmology, deep learning systems (DLS) have been developed to detect various conditions, such as glaucoma,⁹⁻¹² age-related macular degeneration,^{9,13-16} diabetic retinopathy,^{9,17-20} and retinopathy of prematurity.²¹ Within oncology, DL techniques have been applied in diseases, such as breast cancer,^{22,23} glioma,^{8,24} basal cell carcinoma,²⁵ and osteosarcoma.²⁶

One commonality across malignancies is that cancer cell morphology reflects the underlying genetics and careful analysis of cytopathology images often provides, with varying degree of accuracy, helpful prediction for the biological behavior and prognosis of the tumor. However, detailed measurement and analysis of cell morphology features, such as nuclear and nucleolar size, is time-consuming, labor-intensive, and clinically infeasible, thus it is largely limited to a research setting. Analyses of pathology images to extract useful information is ultimately pattern recognition exercise, a task that DL excels in. Using DL to extract useful information from pathology images has been investigated in several diseases. Coudray et al.²⁷ used DL to analyze histopathologic slides to predict the 10 most commonly mutated genes in lung adenocarcinoma. Couture et al.²³ used DL to predict estrogen receptor status in breast tumor pathology slides. Schaumberg et al.²⁸ used DL to predict SPOP mutation state in prostate tumor pathology slides.

DL can be applied in the diagnosis and prognostication of numerous other ocular pathologies. Uveal melanoma (UM), for example, is the most common primary intraocular malignancy in adults.²⁹ UM is unique among malignancies in that gene expression profile (GEP) obtained from fine needle aspiration biopsy (FNAB) samples, independent of other clinicopathological parameters, provides the most accurate prediction currently available for long-term metastasis risk and survival. UM GEP can be divided into two classes: class 1 and class 2, and there is a stark contrast in long-term survival between the two classes—the 92-month survival probability in class 1 patients is 95%, versus 31% in class 2 patients.³⁰

Accordingly, there is a need for additional DL-based image analytical tools, methods, and related aspects, for diagnosing and/or prognosticating ocular pathologies, including UM.

### SUMMARY

The present disclosure relates, in certain aspects, to methods, devices, kits, systems, and computer readable media of use in detecting ocular pathologies. In certain applications, for example, the smart ocular analytical devices and systems disclosed herein capture images of ocular tissues or portions thereof (e.g., cells, organelles, biomolecules, etc.) of a given subject, display those images, and match properties (e.g., patterns or the like) of the captured images with properties of an ocular pathology model that is trained on a plurality of reference images of ocular tissues or portions thereof (e.g., cells, organelles, biomolecules, etc.) of reference subjects. The properties of the ocular pathology model are indicative of at least one pathology. In some implementations, for example, a deep learning system (DLS) is provided that differentiates between GEP class 1 and 2 based on images of disease cells obtained from patients (e.g., cytopathologic samples obtained from FNABs, etc.). These and other aspects will be apparent upon a complete review of the present disclosure, including the accompanying figures.

In one aspect, the present disclosure provides a method of detecting an ophthalmologic genetic disease in a subject at least partially using a computer. The method includes matching, by the computer, one or more properties of one or more images of one or more ocular tissues or portions thereof from the subject with one or more properties of at least one ocular pathology model that is trained on a plurality of reference images of ocular tissues or portions thereof from reference subjects. The properties of the ocular pathology model are indicative of the ophthalmologic genetic disease.

In one aspect, the present disclosure provides a method of classifying uveal melanoma tissues or portions thereof in a subject at least partially using a computer. The method includes matching, by the computer, one or more properties of one or more images of one or more uveal melanoma tissues or portions thereof from the subject with one or more properties of at least one uveal melanoma model that is trained on a plurality of reference images of uveal melanoma tissues or portions thereof from reference subjects. The properties of the uveal melanoma model are indicative of a a survival outcome prediction (e.g., a gene expression profile (GEP) class or the like) of the uveal melanoma.

In another aspect, the present disclosure provides a method of producing an ocular pathology model at least partially using a computer. The method includes dividing, by the computer, reference images of ocular tissues or portions thereof from reference subjects into at least two tiles to generate tile sets, which ocular tissues or portions thereof comprise a given ocular pathology. The method also includes retaining, by the computer, tiles in the tile sets that comprise images of the ocular tissues or portions thereof that comprise the given ocular pathology to generate retained tile sets. In addition, the method also includes inputting, by the computer, the retained tile sets into a neural network comprising a classification layer that outputs survival outcome predictions (e.g., gene expression profile (GEP) classes or the like) for the given ocular pathology to train the neural network, thereby producing the ocular pathology model.

In another aspect, the present disclosure provides a method of treating an ocular pathology of a subject. The method includes capturing one or more images of one or more ocular tissues or portions thereof from the subject that comprise the ocular pathology to generate at least one captured image. The method also includes matching one or more properties of the captured image with one or more properties of at least one ocular pathology model that is trained on a plurality of reference images of ocular tissues or portions thereof from reference subjects, which properties of the ocular pathology model are indicative of the ocular pathology to generate a matched property set. The method also includes classifying the ocular pathology of the subject using the matched property set to generate an ocular pathology classification. In addition, the method also includes administering one or more therapies to the subject based on the ocular pathology classification, thereby treating the ocular pathology of the subject.

In some embodiments of the methods disclosed herein, the ophthalmologic genetic disease comprises cancer (e.g., uveal melanoma). In certain embodiments of the methods disclosed herein, the classification layer comprises a binary classification layer that classifies uveal melanoma samples as gene expression profile (GEP) class 1 or GEP class 2. In some embodiments, the methods disclosed herein include obtaining the ocular tissues or portions thereof from the subject. Typically, the properties comprise one or more patterns.

In some embodiments, the methods disclosed herein also include administering one or more therapies to the subject to treat the ocular pathology. In some embodiments, the methods disclosed herein also include repeating the method at one or more later time points to monitor progression of the ocular pathology in the subject. In some embodiments of the methods disclosed herein, the ocular pathology model comprises one or more selected therapies indexed to the ocular pathology of the subject.

In some embodiments, the methods disclosed herein include capturing the images of the ocular tissues or portions thereof from the subject with a camera. In some embodiments, the camera is operably connected to a database comprising an electronic medical record of the subject. In these embodiments, the method typically further comprises retrieving data from the electronic medical record and/or populating the electronic medical record with at least one of the images and/or information related thereto. In certain embodiments, the camera is wirelessly connected, or connectable, to the electronic medical record of the subject. In some embodiments, the camera and/or the database is wirelessly connected, or connectable, to one or more communication devices of one or more remote users and wherein the remote users view at least one of the images of the ocular tissues or portions thereof of the subject and/or the electronic medical record of the subject using the communication devices. In certain embodiments, the communication devices comprise one or more mobile applications that operably interface with the camera and/or the database. In some embodiments, the users input one or more entries into the electronic medical record of the subject in view of the detected ocular pathology of the subject using the communication devices. In some embodiments, the users order one or more therapies and/or additional analyses of the subject in view of the detected ocular pathology of the subject using the communication devices. In some embodiments, a system that comprises the database automatically orders one or more therapies and/or additional analyses of the subject in view of the detected ocular pathology of the subject when the users input the entries into the electronic medical record of the subject.

In another aspect, the present disclosure provides a system that includes at least one camera that is configured to capture one or more images of ocular tissues or portions thereof from a subject. The system also includes at least one controller that is operably connected, or connectable, at least to the camera. The controller comprises, or is capable of accessing, computer readable media comprising non-transitory computer executable instructions which, when executed by at least one electronic processor, perform at least: capturing the images of the ocular tissues or portions thereof from the subject with the camera to generate captured images, and matching one or more properties of the captured images with one or more properties of at least one ocular pathology model that is trained on a plurality of reference images of ocular tissues or portions thereof of reference subjects, which properties of the ocular pathology model are indicative of at least one ocular pathology.

In another aspect, the present disclosure provides a computer readable media comprising non-transitory computer executable instruction which, when executed by at least electronic processor perform at least: capturing, by a camera, one or more images of ocular tissues or portions thereof from a subject to generate at least one captured image, and matching one or more properties of the captured image with one or more properties of at least one ocular pathology model that is trained on a plurality of reference images of ocular tissues or portions thereof of reference subjects, which properties of the ocular pathology model are indicative of at least one ocular pathology.

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A is a flow chart that schematically depicts exemplary method steps according to some aspects disclosed herein.

FIG. 1B is a diagram that schematically depicts exemplary image processing method steps according to some aspects disclosed herein.

FIG. 2 is a schematic diagram of an exemplary system suitable for use with certain aspects disclosed herein.

FIG. 3 is a schematic representation of data processing. (Panel A) Whole slide scanning; one slide per patient. (Panel B) Snapshot image manually captured at 40×; multiple 40× images were captured from each slide. (Panel C) Each 40× image was further divided into eight tiles of equal sizes.

FIG. 4 show sample CAM analyses of correctly predicted cytopathology images. (Panel A, Patient 5, GEP class 1) The highlighted cells demonstrate classic spindle morphology. Spindle-shaped UM cells are associated with better prognosis and have been shown to correlate with class 1 samples. Panel (B, Patient 10, GEP class 1) The highlighted cells exhibit less atypia than the rest of the cells. Cells with less atypia are associated with a better prognosis and class 1 samples. (Panel C, Patient 13, GEP class 2) The highlighted cell exhibits an epithelioid cytomorphology, which is known to carry a worse prognosis and has been shown to be associated with class 2 samples. (Panel D, Patient 18, GEP class 2) The highlighted region contains a cell with the highest nuclear-cytoplasmic ratio and degree of atypia, features that are associated with a worse prognosis and class 2 classification.

FIG. 5 (Panels A-C) show sample CAM analyses for Patient 6 (GEP class 1), whom the algorithm correctly predicted would have a poor outcome. Note the highlighted heavily pigmented UM cells.

FIG. 6 (Panels A and B) show sample CAM analyses for two GEP class 2 patients, who had unexpectedly extended survival durations after metastasis was detected. The DCNN highlighted the less aggressive cells, with lower nuclear-cytoplasmic ratios and smaller nuclei. (Panel C) show a sample low quality image tile (Patient 15) with copious amount of debris and artefacts that were likely the reasons for failed predictions.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, kits, computer readable media, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent or other therapy to a subject means to give, apply or bring the composition or therapy into contact with or otherwise affect the subject. Administration can be accomplished by any of a number of routes, including, for example, topical, oral, subcutaneous, intramuscular, intraperitoneal, intravenous, intrathecal and intradermal.

Biomolecule: As used herein, “biomolecule” refers to an organic molecule produced by a living organism. Examples of biomolecules, include macromolecules, such as nucleic acids, proteins, carbohydrates, and lipids.

Classifier: As used herein, “classifier” or “classifying” generally refers to algorithm computer code that receives, as input, test data and produces, as output, a classification of the input data as belonging to one or another class (e.g., having a given ocular pathological class).

Detect: As used herein, “detect,” “detecting,” or “detection” refers to an act of determining the existence or presence of one or more pathologies, or properties indicative thereof, in a subject.

Indexed: As used herein, “indexed” refers to a first element (e.g., clinical information) linked to a second element (e.g., a given sample, a given subject, a recommended therapy, etc.).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), support vector machines, decision trees (e.g., recursive partitioning processes such as CART—classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Match: As used herein, “match” means that at least a first value or element is at least approximately equal to at least a second value or element. In certain embodiments, for example, one or more properties of a captured image (e.g., patterns or the like within the image) from a test subject are used to detect a pathology in the test subject when those properties are at least approximately equal to one or more properties of an ocular pathology model.

Ocular Tissues Or Portions Thereof: As used herein, “ocular tissues or portions thereof” refer to tissues, cells, organelles, and/or biomolecules from the ocular system of a subject.

Ocular Pathology Model: As used herein, “ocular pathology model” refers to a computer algorithm or implementing system that performs ophthalmologic detections, diagnoses, decision-making, prognostication, and/or related tasks that typically rely solely on expert human intelligence (e.g., an ophthalmologist or the like). In some embodiments, an ocular pathology model is produced using reference images of ocular tissues or portions thereof and/or videos as training data, which is used to train a machine learning algorithm or other artificial intelligence-based application. In some implementation, the model comprises an “uveal melanoma model.”

Ophthalmologic Genetic Disease: As used herein, “ophthalmologic genetic disease” refers to a disease, condition, or disorder of the ocular system of a subject that is caused by one or more abnormalities (e.g., mutations) in the genome of that subject.

Pathology: As used herein, “pathology” refers to a deviation from a normal state of health, such as a disease (e.g., neoplastic or non-neoplastic diseases), abnormal condition, or disorder.

Reference Images: As used herein, “reference images” or “reference videos” refer a set of images and/or videos (e.g., a sequence of images) having or known to have or lack specific properties (e.g., known pathologies in associated subjects and/or the like) that is used to generate ocular pathology models (e.g., as training data) and/or analyzed along with or compared to test images and/or videos in order to evaluate the accuracy of an analytical procedure. A set of reference images typically includes from at least about 25 to at least about 10,000,000 or more reference images and/or videos. In some embodiments, a set of reference images and/or videos includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more reference images and/or videos.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., known ocular or other pathology and/or the like).

### DETAILED DESCRIPTION

With millions of patients affected per year, ocular pathologies are a leading diagnosis for health care visits in the U.S. However, healthcare providers often have uncertainty in identifying eye-related diseases, disorders, or conditions. Uncertainty of the eye exam stems from its small, complex anatomy, and the complexity and cost of traditional ocular diagnostic/prognostic approaches that makes learning and mastering ocular exams challenging. Accordingly, in certain aspects, the present disclosure provide deep learning methods of analyzing ocular cytopathology images to detect a given ocular pathology in a subject, predict a likely outcome for the subject, and/or determine the genetic profile of the subject.

To address the limitations of the pre-existing technology, the present disclosure provides an artificial intelligence (AI)-based image analysis systems of use in diagnosing and managing ocular pathologies in certain embodiments. In some implementations, the present disclosure also relates to mobile applications (apps) that feature image recognition using machine learning algorithms to give a diagnosis, or at least an AI augmented diagnosis, of an eye exam and provide management recommendations to healthcare providers and other users. A digital image of the eye exam or an ocular tissue or cell sample from the exam, aided by the diagnosis provided by the mobile app, improves provider certainty of the diagnosis and prognostication, among other attributes. In some embodiments, the present disclosure provides ocular devices and systems that are configured for digital image capture and data analysis in addition to having connectivity (e.g., wireless connectivity) to patients' electronic medical records (EMRs). The smart ocular analysis systems disclosed herein enable users, irrespective of their level of training or experience, the ability to identify and treat ocular pathologies with the precision of an ocular specialist (e.g., an ophthalmologist) and to otherwise improve diagnostic accuracy and ocular disease management. Some embodiments disclosed herein emphasize the analysis of uveal melanoma in subjects. However, it will be appreciated that the present disclosure can be applied in the diagnosis and prognostication of numerous other ocular pathologies.

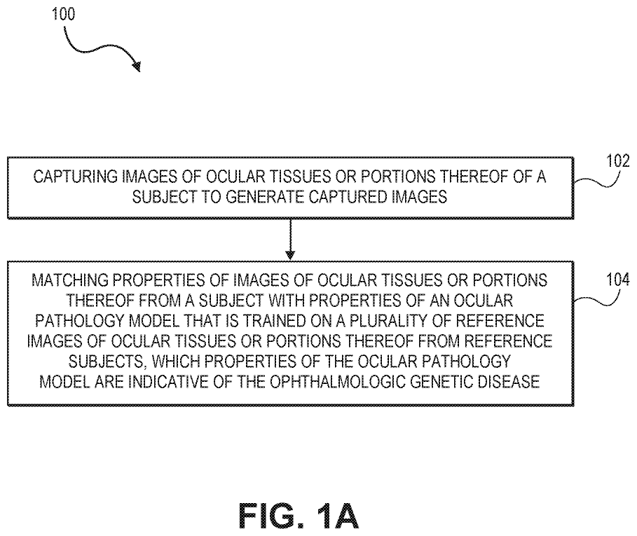

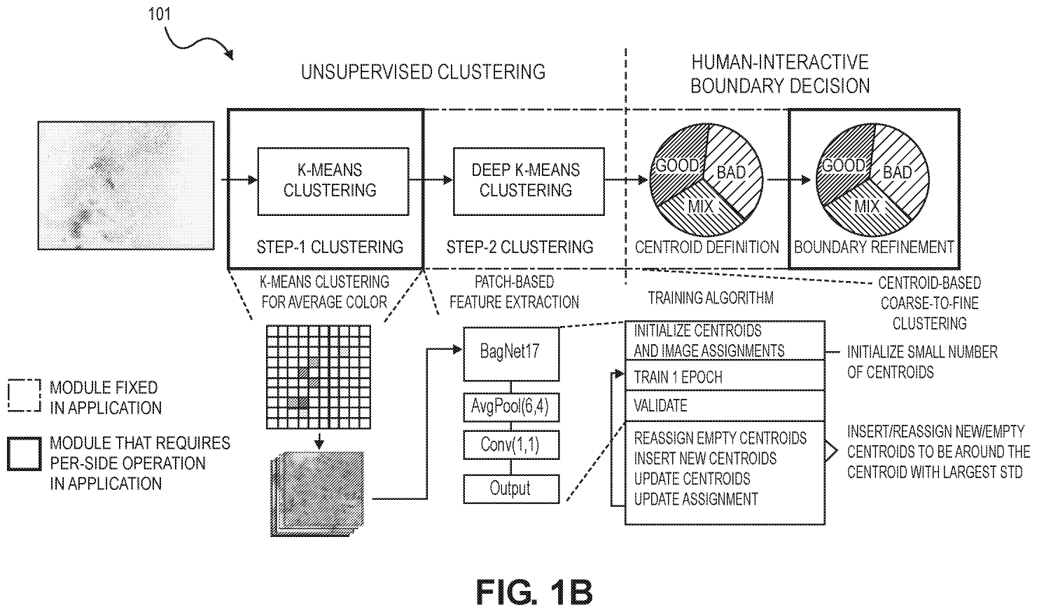

To illustrate, FIG. 1A is a flow chart that schematically depicts exemplary method steps according to some aspects disclosed herein. As shown, method 100 is for detecting includes an ophthalmologic genetic disease in a subject and includes capturing images of ocular tissues or portions thereof of the subject to generate captured images. Typically, this process includes obtaining a sample of the ocular tissues or portions thereof from the subject (e.g., via a fine needle aspiration biopsy procedure or the like) (step 102). Those samples are generally positioned on one or more microscope slides for image capture. Essentially any type of camera is adapted for use in generating the images utilized as part of the processes described herein. In some of these embodiments, for example, whole slides are scanned (e.g., at a magnification of about 40× or another magnification level suitable to capture an image of an entire slide in a single scan) using an Aperio ScanScope AT machine [Wetzlar, Germany] or the like. Method 100 also includes matching properties of images of ocular tissues or portions thereof from the subject with properties of an ocular pathology model that is trained on a plurality of reference images of ocular tissues or portions thereof from reference subjects. The properties of the ocular pathology model are indicative of the ophthalmologic genetic disease (e.g., uveal melanoma or another type of ocular cancer) (step 104). In some embodiments, the steps of capturing an image of a given test subject's sample and matching properties of the test subject's image with those of the ocular pathology model are performed in substantially real-time during a given examination procedure. In some embodiments, images are captured directly from a test subject's eye and properties (e.g., patterns or the like) of those images are matched with those of the ocular pathology model to provide a diagnostic and/or prognostic determination.

The ocular pathology model utilized with the methods and related aspects disclosed herein are generated using various approaches. In some embodiments, for example, an ocular pathology model is generated using large datasets of reference images of ocular tissues or portions thereof from reference subjects (disposed on slides), which ocular tissues or portions thereof comprise a given ocular pathology. Each reference image of a given slide is typically divided into two or more tiles to generate two or more tile sets. Only tiles in the tile sets that include images of diseased ocular tissues or portions thereof from the referenced subjects are typically retained to generate retained tile sets. In these embodiments, the method also generally includes inputting the retained tile sets into a neural network that includes a classification layer that outputs survival outcome predictions (e.g., gene expression profile (GEP) classes or the like) for the given ocular pathology to train the neural network for use as an ocular pathology model. In certain embodiments, the classification layer is a binary classification layer that classifies uveal melanoma samples as GEP class 1 or GEP class 2 and/or another survival outcome prediction. The ocular pathology model is typically trained on a plurality of reference images and/or videos (e.g., about 50, about 100, about 500, about 1,000, about 10,000, or more reference images and/or videos) of ocular tissues or portions thereof of reference subjects.

The devices and systems disclosed herein also generally include a controller (e.g., a local processor, etc.) at least partially disposed within device and system body structures. A controller is general operably connected the camera (e.g., disposed within the camera structure in certain embodiments) and to a display screen, in certain embodiments. In addition, the controller typically includes, or is capable of accessing (e.g., remotely via a wireless connection), computer readable media (e.g., embodying an artificial intelligence (AI)-based algorithm) comprising non-transitory computer executable instructions which, when executed by at least one electronic processor, perform capturing images and/or videos of the ocular tissues or portions thereof of a subject, and displaying the captured images and/or videos on the display screen. The computer executable instructions also perform matching one or more properties (e.g., test pixel or other image patterns) of the captured images and/or videos with one or more properties e.g., reference pixel or other image patterns) of an ocular pathology model that is trained on a plurality of reference images and/or videos (e.g., about 50, about 100, about 500, about 1,000, about 10,000, or more reference images and/or videos) of diseased ocular tissues or portions thereof of reference subjects. The properties of the ocular pathology model are typically indicative of at least one ocular-related pathology (e.g., cancer, age-related macular degeneration (AMD), cataracts, CMV retinitis, diabetic macular edema (DME), glaucoma, ocular hypertension, uveitis, etc.). Ocular pathologies are also described in, for example, Yanoff et al., Ocular Pathology, 7th Edition, Elsevier (2014). The ocular pathology models disclosed herein are typically generated using one or more machine learning algorithms. In some of these embodiments, the machine learning algorithms include one or more neural networks. In certain embodiments, ocular pathology models include selected therapies indexed to a given ocular pathology to provide therapy recommendations to healthcare providers or other users when the pathology is detected in a subject.

The controllers of the devices and systems disclosed herein include various embodiments. In some embodiments, for example, the controller of a given device is wirelessly connected, or connectable, to one or more of the computer executable instructions. In certain embodiments, the controller is operably connected, or connectable, to a database that includes electronic medical records (EMRs) of subjects. In these embodiments, the computer executable instructions typically further perform retrieving data from the electronic medical record and/or populating the electronic medical record with at least one of the images and/or videos, selected smart phrases, and/or other related information. In certain of these embodiments, the controller is wirelessly connected, or connectable, to the electronic medical records. Typically, the device, system, and/or the database is wirelessly connected, or connectable, to one or more communication devices (e.g., mobile phones, tablet computers, etc.) of remote users. This enables the remote users to view the captured images and/or videos of the sample of a given subject and/or the electronic medical record of that subject using the communication devices. In some of these embodiments, the communication devices include one or more mobile applications that operably interface with the devices, systems, and/or the database. In these embodiments, the remote users are generally capable of inputting entries into the electronic medical record of the subject in view of a detected ocular pathology of the subject using the communication devices. In some of these embodiments, the users are capable of ordering one or more therapies and/or additional analyses of the subject in view of the detected pathology of the subject using the communication devices.

In some embodiments, the ocular analytical devices or systems of the present disclosure are provided as components of kits. Various kit configurations are optionally utilized, but in certain embodiments, one or more devices or system are packaged together with computer readable media, replacement lenses, replacement illumination sources (e.g., LEDs, etc.), rechargeable battery charging stations, batteries, operational instructions, and/or the like.

In some embodiments, method 100 is repeated at one or more later time points to monitor progression of the pathology in the subject. In certain embodiments, method 100 includes administering one or more therapies to the subject to treat the pathology. In some of these embodiments, remote users (e.g., healthcare providers) order the therapies and/or additional analyses of the subject in view of the detected ocular pathology in the subject using a communication device, such as a mobile phone or remote computing system. In certain of these embodiments, a system that comprises the database automatically orders the therapies and/or additional analyses of the subject in view of the detected ocular pathology of the subject when remote users input the entries into the electronic medical record of the subject. Additional aspects of methods of using the ocular devices and systems are described herein.

To further illustrate, in some embodiments, to develop an artificial intelligence algorithm for pathology slide analysis, the data from a pathology slide is extracted first. In these embodiments, a glass pathology slide is typically captured digitally using whole slide imaging. Processing a whole slide image poses at least two unique challenges that utilize customized solutions, as described herein. First, each slide generally contains a massive amount of information that is broken down into an appropriate or manageable data package size. Second, regions of interest (ROI) are differentiated from irrelevant or unusable regions. Both tasks can be performed manually, which is labor-intensive, time-consuming, costly and thus generally infeasible. Hence, in certain embodiments, a human-assisted computation tool is provided that enables large-scale, efficient processing of digital whole slide imaging. In these embodiments, the overall technical pipeline can be divided into two general stages: unsupervised clustering and human-interactive boundary decision (FIG. 1B). Each of these steps of method 101 is described separately below.

Step-1 Clustering

As shown in this exemplary embodiment, a whole slide image is first down-sampled, such that each pixel in the resultant image corresponds to the average signal within one area. The size of this area is only constrained by its compatibility with the following clustering steps. The area of 512×512 pixel performs sufficiently well in some embodiments. K-means clustering is then typically used to cluster pixel intensities into two centroids that intuitively correspond to regions with bright and dark average intensities. Since whole slide images are acquired with the bright-field technique in some embodiments, pixels with low and high intensities correspond to regions with high and low tissue content, respectively. This method is typically used to screen out the empty/blank patches. Because the exact magnitude of bright and dark centroid intensities varies with cell distribution and density, this clustering scheme is typically applied to every pathology slide independently.

Step-2 Clustering

Step-2 clustering generally aims to separate high-quality images with usable information from low-quality images that either contain insufficient information or artifacts. Since this separation is typically based on image content that can vary considerably across pixels, clustering is often performed on 228×228 pixel ROIs in naive resolution, which are much smaller than the areas extracted from Step-1 clustering. These patches are extracted with a stride of 128 from the ROIs selected in Step-1 clustering in some embodiments. This step of clustering is typically performed using a deep neural network or another machine learning algorithm.

Human-Interactive Boundary Decision

In these embodiments, after Step-2 clustering, every centroid typically contains ROIs that exhibit similar appearance. However, at this point it is often still unclear which of the ROIs in the centroids are high- and low-quality. To provide this semantic definition with minimal manual annotation, a Graphical User Interface (GUI) is used in some embodiments that allows for rapid centroid annotation by a human expert. To this end, 10 ROIs from 10 random centroids are displayed for the user to classify in some of these embodiments. After several iterations, each centroid has more than 10 high-/poor-quality annotations. The number of high- and poor-quality ROIs classified to every centroid is then used to define a decision boundary that separates between high- and low-quality ROIs. To allow for the refinement of ROI suggestions, a patient-specific refinement tool is created that visualizes ROI assignments based on the previous centroid-based classification. As shown in method 101, high-/low-/mix-quality assignments are shown together and synchronized with the corresponding whole slide image in these embodiments. The user can hover the mouse to display the underlying ROI in native resolution, and can simply click the ROI to re-annotate if necessary. In this exemplary case, the selected ROI and all ROIs in the surrounding area in the feature space are all re-annotated.

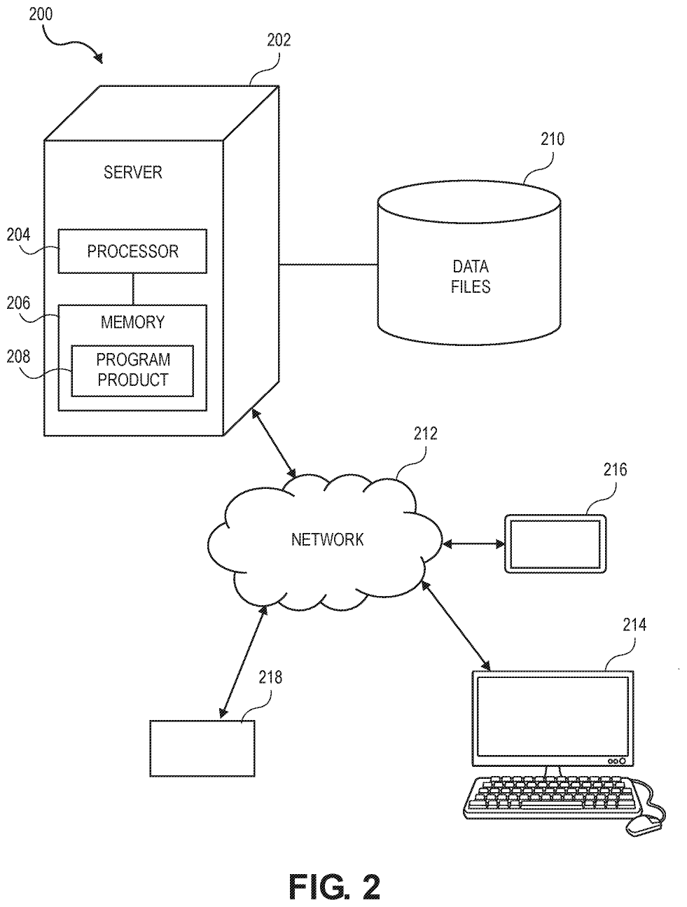

The present disclosure also provides various deep learning systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving captured images and/or videos for further analysis, etc.)) positioned remote from camera device 218, and in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., related to an ocular pathology model) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to more reference images and/or videos, indexed therapies, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 508 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one captured image, EMR, and/or the like to be displayed (e.g., via camera 218 and/or via communication devices 214, 216 or the like) and/or receive information from other system components and/or from a system user (e.g., via camera 218 and/or via communication devices 214, 216, or the like).

In some aspects, program product 208 includes non-transitory computer-executable instructions which, when executed by electronic processor 204 perform at least: capturing, by a camera, one or more images of ocular tissues or portions thereof from a subject to generate a captured image, and matching one or more properties of the captured image with one or more properties of at least one ocular pathology model that is trained on a plurality of reference images of ocular tissues or portions thereof of reference subjects, which properties of the ocular pathology model are indicative of at least one pathology. Other exemplary executable instructions that are optionally performed are described further herein.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### Example

Methods

Dataset

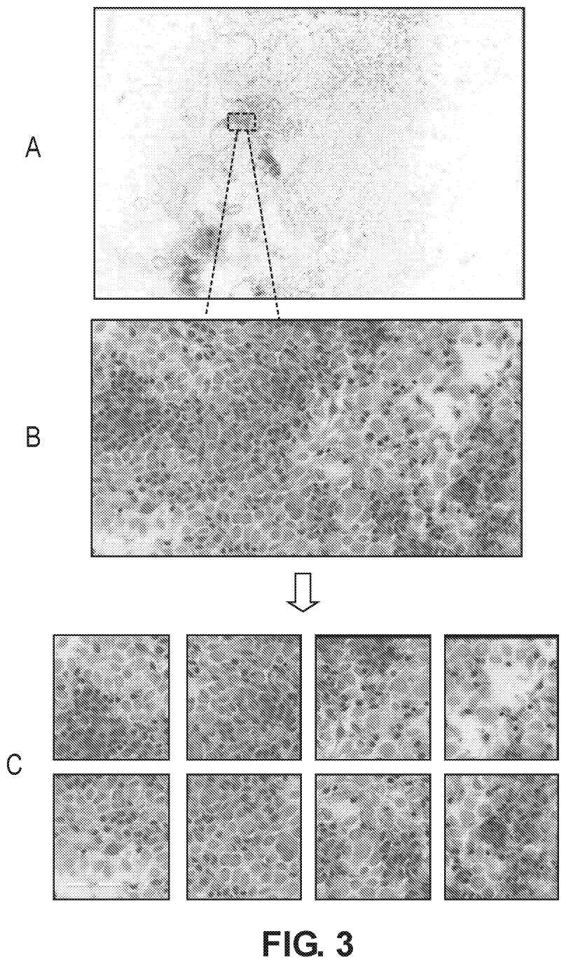

In total, 20 de-identified FNAB cytology slides from 20 patients with UM (one slide per patient) were included in this study. The FNAB was performed as standard clinical care to confirm the diagnosis of UM and to obtain cellular material for genetic analysis. The cytology specimen was flushed on a standard pathology glass slide, smeared, and stained with hematoxylin and eosin (H&E). The specimen submitted for GEP was flushed into a tube containing extraction buffer and submitted for DecisionDx-UM® testing [Friendswood, Tex.]. Of the 20 specimens, 10 belonged to GEP Class 1 and 10 belonged to GEP Class 2. Whole-slide scanning was performed for each cytology slide at a magnification of 40×, using the Aperio ScanScope AT machine [Wetzlar, Germany], and the high-magnification digital image was examined using the Aperio Imagescope® software. Using a magnification of 40×, snapshot images containing melanoma cells were saved in a TIFF format. Each snapshot image measured 1,716 pixels (width)×926 pixels (height), and further split into 8 tiles of equal size. The tiles were then examined, and only tiles consisting of at least one melanoma cell were saved. Out of the 20 slides, a total of 26,351 unique tiles were generated. The data was processed such that the final image tiles fit the input size dimension of the deep convolutional neural network (DCNN). Schematic representation for data processing is shown in FIG. 3.

Deep Learning System Development

Using transfer learning, the study adopted a readily available ResNet-152³¹ DCNN that was pre-trained on ImageNet.³² The last fully connected layer of ResNet-152 was redefined to have 2 outputs for the underlying binary classification problem, distinguishing class 1 from class 2 patients. After convergence of training of the last fully connected layer, all parameters were unfrozen and adapted with lower learning rate to avoid “forgetting”. By the end of the training/validation phases, the weights that attained the optimal validation accuracy were set as the model parameters.

Model Performance Evaluation

“Leave-one-out” cross-validations were performed to evaluate the performance of the DLS. To test each of the 20 slides/patients, 10 models were trained using different training/validation split. That is, for each of the leave-one-out cross-validation, 10 random samplings were performed for the validation subset selection. If “slide 1” was used as the testing slide, then the other 19 slides were used for model development: 17 slides for training and 2 slides for validation (one from class 1 and one from class 2). “Slide 1” was then tested 10 different times by 10 different models that were generated by 10 random and different combinations of training and validation slides. For example, model #1 would use “slide 2” and “slide 11” for validation. Model #2 would use “slide 3” and “slide 12” for validation. Model #3 would use “slide 4” and “slide 13” for validation, etc. Eventually, 10 models were generated, and the mean accuracy of these 10 models was obtained. If the lower 95 confidence interval (CI) value exceeded 50%, then it was a concluded that the GEP of “slide 1” (patient 1) was correctly predicted. This process was repeated for all 20 slides/patients, such that each slide/patient was evaluated 10 times by 10 different models. This evaluation method was adopted to account for the fact that due to the low amount of data variation, the validation slides would have a strong effect on the model performance.

Heat Map Generation

To identify features in the images used by the DCNN to predict GEP, heatmaps were created through class activation mapping (CAM)³³, a technique that visually highlights areas of importance in terms of classification decision within an image (the “warmer” the color, e.g., red, the more important is a particular feature). This technique was chosen for its ability to convey information in a visually vivid manner. The original image was preserved, allowing all the image features to remain present, and the overlaid color spectrum provided a clear linear scale of feature importance.

### Results

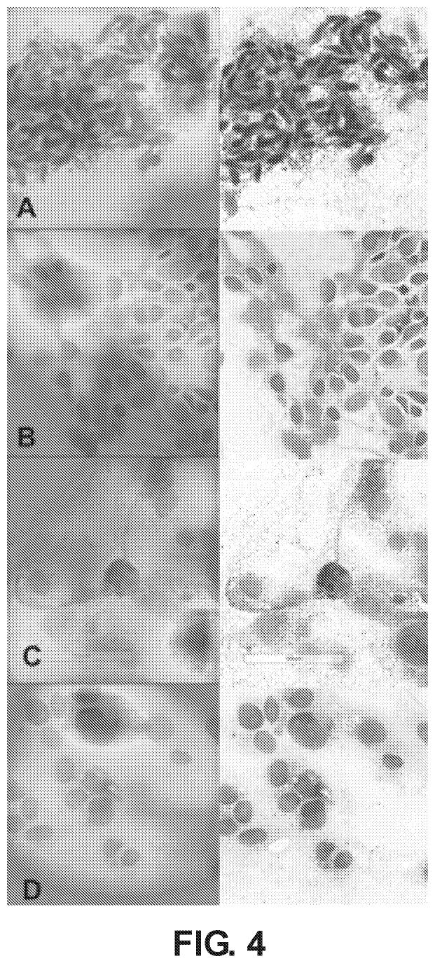

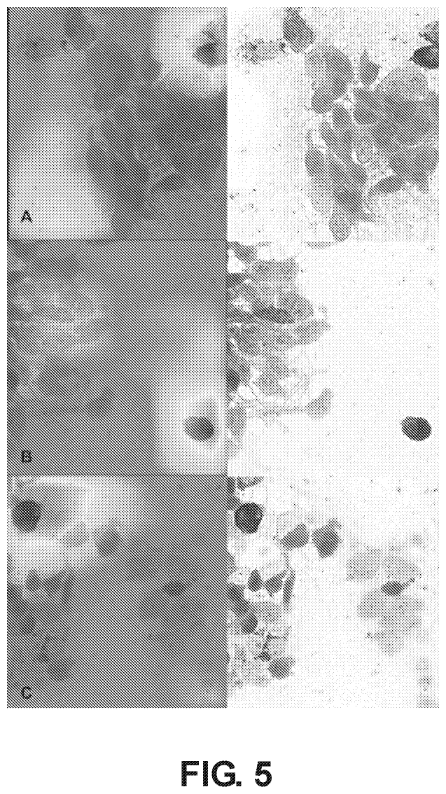

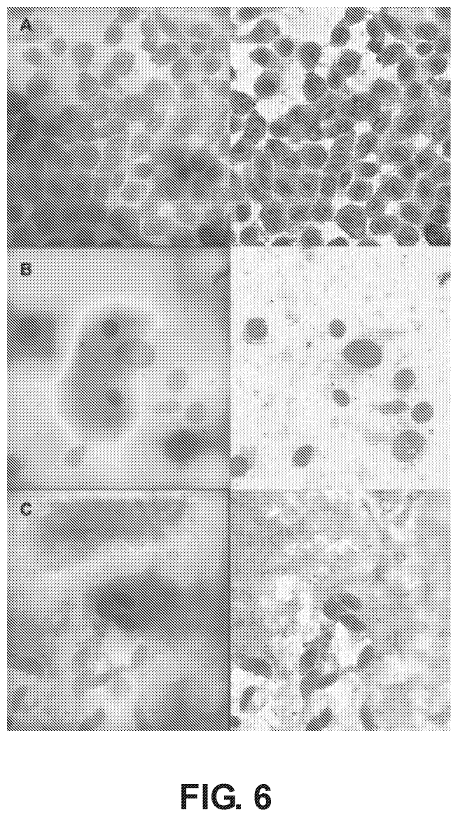

This study was able to predict the GEP in 15/20 (75%) of the cohort of UM patients. The mean and 95 CI accuracy % for each patient are summarized in Table 1. One patient (patient 17, class 2) received equivocal prediction from the model. She also died of an unrelated breast cancer 19 months after her diagnosis of UM, so her UM-specific survival outcome could not be ascertained. Four patients received opposite GEP predictions from the model: patient 6 (class 1), patient 11 (class 2), patient 12 (class 2) and patient 15 (class 2). The detailed clinical information of the 4 patients, who received opposite survival predictions, is summarized in Table 2 and is further discussed in the discussion section. CAM analyses were performed on image tiles derived from 8 patients: 4 patients whose GEP was correctly predicted (FIG. 4) and 4 patients whose GEP was incorrectly predicted (FIGS. 5 and 6). Each image tile usually contained numerous UM cells, but CAM analyses typically only showed activation centered on a small subset of cells within each tile.

TABLE 1
GEP prediction accuracy in the uveal melanoma patients.

| Patient # | GEP Class | Mean | Upper 95 Cl | Lower 95 Cl |
|---|---|---|---|---|
| 1 | 1 | 82.4% | 89.5% | 75.3% |
| 2 | 1 | 84.9% | 90.3% | 79.4% |
| 3 | 1 | 63.3% | 71.8% | 54.7% |
| 4 | 1 | 81.0% | 89.8% | 72.3% |
| 5 | 1 | 96.2% | 98.9% | 93.5% |
| 6 | 1 | 30.8% | 35.0% | 26.6% |
| 7 | 1 | 98.7% | 99.6% | 97.9% |
| 8 | 1 | 92.8% | 100.0% | 85.4% |
| 9 | 1 | 80.8% | 83.0% | 78.7% |
| 10 | 1 | 95.6% | 99.2% | 91.9% |
| 11 | 2 | 27.1% | 38.8% | 15.5% |
| 12 | 2 | 35.9% | 44.2% | 27.5% |
| 13 | 2 | 98.1% | 99.3% | 96.8% |
| 14 | 2 | 65.3% | 77.9% | 52.6% |
| 15 | 2 | 13.4% | 16.9% | 9.9% |
| 16 | 2 | 98.7% | 99.5% | 97.9% |
| 17 | 2 | 57.8% | 72.8% | 42.9% |
| 18 | 2 | 99.1% | 99.5% | 98.7% |
| 19 | 2 | 71.1% | 80.9% | 61.3% |
| 20 | 2 | 64.6% | 74.8% | 54.4% |

Mean, upper 95 CI and lower 95 CI accuracy % for each patient generated by leave-one-out cross validations. An accurate prediction is defined as >50% accuracy for both the upper and lower 95 CI value.

TABLE 2
Table 2. Clinical outcomes of UM patients who received an
opposite survival prediction by our deep learning system.

| | | | | Months | | | | |
|---|---|---|---|---|---|---|---|---|
| | | | | between | Months | | | |
| | | | Age at | diagnosis | between | | | |
| Patient | GEP | | diagnosis | and | metastasis | CB | LBD | Thickness |
| # | class | Gender | (year) | metastasis | and death | involvement | (mm) | (mm) |
| 6 | 1 | F | 80.6 | 25 | 3 | Y | 19 | 5.5 |
| 11 | 2 | M | 48.9 | 19 | 20 | Y | 10.5 | 4.3 |
| 12 | 2 | F | 62 | 0 | at least 23 | N | 18 | 11.5 |
| 15 | 2 | M | 79.1 | ~12 | ~12 | Y | 16 | 8 |

GEP = gene expression profile; CB = ciliary body; LBD = largest basal diameter.

### Discussion

Under the hypothesis that DL methods, when applied appropriately in cytopathology image analysis, could predict a UM's prognosis, it was set out to develop a DLS that can differentiate between GEP class 1 and class 2, based on FNAB cytology slides, given the close correlation between GEP and survival in UM patients. On a patient level, the study was able to predict the GEP status in 75% of the cohort.

Sample CAM analyses for the correctly-predicted images showed that the DCNN was able to focus on biologically-relevant features to make the correct predictions. For GEP class 1 images, the DCNN generally focused on UM cells with spindle-shaped morphology or less atypia (FIGS. 4A and B), features that are associated with a better prognosis and class 1 classification. For GEP class 2 images, the DCNN generally focused on UM cells with epithelioid morphology, more atypia, larger nuclei and larger nucleoli (FIGS. 4C and D), features that are associated with worse survivial.³⁴⁻³⁶

Four of the 20 patients received an opposite prediction, and these 4 cases will be discussed in detail in the following.

Patient 6's UM was classified as GEP class 1. The tumor was broad with a largest basal diameter (LBD) of 19 mm. She died of metastatic UM 28 months after her initial diagnosis. Although LBD has been shown to be an important prognostic factor independent of GEP,³⁷ the clinical course of this patient was certainly much worse than expected, which was correctly predicted by the algorithm. On review of the CAM analyses, it was noticed that on multiple occasions the algorithm focused on UM cells containing copious amount of melanin (FIG. 5). This was in line with the observation made by McLean et al.³⁸ that heavy pigmentation was associated with more aggressive tumor behavior.

Patient 11's UM was classified as GEP class 2. He died of metastatic UM 39 months after his initial diagnosis, but he survived for 20 months after metastasis was detected. Patient 12's UM was classified as GEP class 2, and the tumor was both broad (LBD of 18 mm) and thick (11.5 mm). She was diagnosed with metastatic UM at presentation, but survived for at least 23 months. The algorithm predicted these 2 patients to have a favorable diagnosis. Although both patients did develop metastasis, their clinical outcomes were certainly much better than the average patient with metastatic UM. For comparison, the median survival time after metastasis diagnosis and the overall 1-year survival rate for metastatic UM has been reported to be 3.9 months and 21.2%, respectively.³⁹ CAM analyses for these 2 patients showed our DCNN generally focusing on less aggressive UM cells within each image tile (FIGS. 6A and B).

Lastly, patient 15's UM was classified as GEP class 2. He was diagnosed with metastasis approximately 12 months after his initial diagnosis, and died of metastatic UM approximately 12 months after metastasis detection. His clinical course was typical for a GEP class 2 tumor, so the algorithm simply failed to make the correct prediction. The image tiles generated from this patient contained copious amount of debris, on which the DCNN often focused (FIG. 6C). The presence of debris and artefacts may have contributed to the generation of wrong predictions.

In summary, the algorithm was able to predict GEP in the cohort of UM patients, with a reasonable accuracy of 75%. Given GEP is highly correlated with survival, the study suggests that prognostication information can be predicted from H&E pathology slides alone in UM using DL. Of particular interests are the opposite predictions made by the algorithm. The algorithm was able to predict poor outcome in a class 1 patient who had an unexpected early death due to metastatic disease. If reproduced in multiple patients in a prospective fashion, such ability to predict unfavorable clinical surprises will be immensely valuable, as it could lead to better surveillance recommendations, earlier detection of metastasis and possible improved survival in the future when more effective treatments for metastatic UM become available. In addition, the algorithm predicted a “favorable” outcome in two class 2 patients, who survived for >20 months after metastasis was detected, significantly longer than the median survival time of 3.9 months in similar patients. This suggests that the algorithm may be able to provide more fine-grained survival prediction in class 2 patients. These observations offer the exciting possibility that a more mature version of the algorithm, trained with a larger dataset and validated prospectively, can further serve as a survival prediction tool, which can be performed remotely and will be more efficient and cost-effective than the current gold standard GEP test which is not available outside of the United States. Alternatively, the algorithm can serve as an enhancement to the current GEP test, by fine-tuning survival prediction and predicting unfavorable clinical surprises in class 1 patients.

The study has several limitations. First, the method reported in the current study obtained cytology samples of UM through FNABs. FNABs may not be possible in certain scenarios, such as in tumors that are very thin. FNABs are also technically challenging, and can yield insufficient material for cytopathologic classification in up to 21.9% of cases even in the hands of experienced ocular oncologists.⁴⁰ Second, due to the limitations of the currently available saliency analysis techniques, the algorithm is only partially explainable. For example, within an image tile with both spindle and epithelioid UM cells or within an image tile with cells of varying degree of atypia, it is unclear how the algorithm decides which cells to focus on and makes a prediction accordingly. Also, the algorithm may be susceptible to the presence of debris and artefacts captured in the pathology images. Third, although the DLS was developed with >25,000 unique data points, it ultimately only included data from 20 UM patients. The small patient sample size and data variation necessitated the use of leave-one-out validations, instead of the more conventional one-shot models. Also, the low data variation likely limits the generalizability of the model.

### REFERENCES

1. LeCun Y, Bengio Y, Hinton G. Deep learning. Nature. 2015; 521(7553):436-444.
2. Saltz J, Gupta R, Hou L, et al. Spatial Organization and Molecular Correlation of Tumor-Infiltrating Lymphocytes Using Deep Learning on Pathology Images. Cell Rep. 2018; 23(1):181-193 e187.
3. Sirinukunwattana K, Ahmed Raza S E, Yee-Wah T, Snead D R, Cree I A, Rajpoot N M. Locality Sensitive Deep Learning for Detection and Classification of Nuclei in Routine Colon Cancer Histology Images. IEEE Trans Med Imaging. 2016; 35(5):1196-1206.
4. Xu J, Luo X, Wang G, Gilmore H, Madabhushi A. A Deep Convolutional Neural Network for segmenting and classifying epithelial and stromal regions in histopathological images. Neurocomputing. 2016; 191:214-223.
5. Chung S W, Han S S, Lee J W, et al. Automated detection and classification of the proximal humerus fracture by using deep learning algorithm. Acta Orthop. 2018; 89(4):468-473.
6. Lakhani P, Sundaram B. Deep Learning at Chest Radiography: Automated Classification of Pulmonary Tuberculosis by Using Convolutional Neural Networks. Radiology. 2017; 284(2):574-582.
7. Lakhani P. Deep Convolutional Neural Networks for Endotracheal Tube Position and X-ray Image Classification: Challenges and Opportunities. J Digit Imaging. 2017; 30(4):460-468.
8. Chang P, Grinband J, Weinberg B D, et al. Deep-Learning Convolutional Neural Networks Accurately Classify Genetic Mutations in Gliomas. AJNR Am J NeuroradioL 2018; 39(7):1201-1207.
9. Ting D S W, Cheung C Y, Lim G, et al. Development and Validation of a Deep Learning System for Diabetic Retinopathy and Related Eye Diseases Using Retinal Images From Multiethnic Populations With Diabetes. JAMA. 2017; 318(22):2211-2223.
10. Asaoka R, Murata H, Iwase A, Araie M. Detecting Preperimetric Glaucoma with Standard Automated Perimetry Using a Deep Learning Classifier. Ophthalmology. 2016; 123(9):1974-1980.
11. Cerentini A, Welfer D, Cordeiro d'Ornellas M, Pereira Haygert C J, Dotto G N. Automatic Identification of Glaucoma Using Deep Learning Methods. Stud Health Technol Inform. 2017; 245:318-321.
12. Muhammad H, Fuchs T J, De Cuir N, et al. Hybrid Deep Learning on Single Wide-field Optical Coherence tomography Scans Accurately Classifies Glaucoma Suspects. J Glaucoma. 2017; 26(12)1086-1094.
13. Burlina P, Pacheco K D, Joshi N, Freund D E, Bressler N M. Comparing humans and deep learning performance for grading AMD: A study in using universal deep features and transfer learning for automated AMD analysis. Comput Biol Med. 2017; 82:80-86.
14. Burlina P M, Joshi N, Pekala M, Pacheco K D, Freund D E, Bressler N M. Automated Grading of Age-Related Macular Degeneration From Color Fundus Images Using Deep Convolutional Neural Networks. JAMA Ophthalmol. 2017; 135(11):1170-1176.
15. Matsuba S, Tabuchi H, Ohsugi H, et al. Accuracy of ultra-wide-field fundus ophthalmoscopy-assisted deep learning, a machine-learning technology, for detecting age-related macular degeneration. Int Ophthalmol. 2018.
16. Treder M, Lauermann J L, Eter N. Automated detection of exudative age-related macular degeneration in spectral domain optical coherence tomography using deep learning. Graefes Arch Clin Exp Ophthalmol. 2018; 256(2):259-265.
17. Gulshan V, Peng L, Coram M, et al. Development and Validation of a Deep Learning Algorithm for Detection of Diabetic Retinopathy in Retinal Fundus Photographs. JAMA. 2016; 316(22):2402-2410.
18. Gargeya R, Leng T. Automated Identification of Diabetic Retinopathy Using Deep Learning. Ophthalmology. 2017; 124(7):962-969.
19. Raju M, Pagidimarri V, Barreto R, Kadam A, Kasivajjala V, Aswath A. Development of a Deep Learning Algorithm for Automatic Diagnosis of Diabetic Retinopathy. Stud Health Technol Inform. 2017; 245:559-563.
20. Takahashi H, Tampo H, Arai Y, Inoue Y, Kawashima H. Applying artificial intelligence to disease staging: Deep learning for improved staging of diabetic retinopathy. PLoS One. 2017; 12(6):e0179790.
21. Brown J M, Campbell J P, Beers A, et al. Automated Diagnosis of Plus Disease in Retinopathy of Prematurity Using Deep Convolutional Neural Networks. JAMA Ophthalmol. 2018; 136(7):803-810.
22. Ehteshami Bejnordi B, Veta M, Johannes van Diest P, et al. Diagnostic Assessment of Deep Learning Algorithms for Detection of Lymph Node Metastases in Women With Breast Cancer. JAMA. 2017; 318(22):2199-2210.
23. Couture H D, Williams L A, Geradts J, et al. Image analysis with deep learning to predict breast cancer grade, E R status, histologic subtype, and intrinsic subtype. NPJ Breast Cancer. 2018; 4:30.
24. Ertosun M G, Rubin D L. Automated Grading of Gliomas using Deep Learning in Digital Pathology Images: A modular approach with ensemble of convolutional neural networks. AMIA Annu Symp Proc. 2015; 2015:1899-1908.
25. Cruz-Roa A A, Arevalo Ovalle J E, Madabhushi A, Gonzalez Osorio F A. A deep learning architecture for image representation, visual interpretability and automated basal-cell carcinoma cancer detection. Med Image Comput Comput Assist Interv. 2013; 16(Pt 2):403-410.
26. Mishra R, Daescu O, Leavey P, Rakheja D, Sengupta A. Convolutional Neural Network for Histopathological Analysis of Osteosarcoma. J Comput Biol. 2018; 25(3):313-325.
27. Coudray N, Ocampo P S, Sakellaropoulos T, et al. Classification and mutation prediction from non-small cell lung cancer histopathology images using deep learning. Nat Med. 2018; 24(10):1559-1567.
28. Schaumberg, A. J., Rubin, M. A., Fuchs, T. J., 2016. H&e-stained whole slide deep learning predicts SPOP mutation state in prostate cancer.
29. Singh A D, Turell M E, Topham A K. Uveal melanoma: trends in incidence, treatment, and survival. Ophthalmology. 2011; 118(9):1881-1885.
30. Onken M D, Worley L A, Ehlers J P, Harbour J W. Gene expression profiling in uveal melanoma reveals two molecular classes and predicts metastatic death. Cancer Res. 2004; 64(20):7205-7209.
31. He, K., Zhang, X., Ren, S., Sun, J. Deep Residual Learning for Image Recognition. The IEEE Conference on Computer Vision and Pattern Recognition (CVPR), 2016, pp. 770-778.
32. Deng, J., Dong, W., Socher, R. et al. Imagenet: A large-scale hierarchical image database. The IEEE Conference on Computer Vision and Pattern Recognition (CVPR), 2009, pp. 248-255.
33. Bolei Zhou, Aditya Khosla, Agata Lapedriza, Aude Oliva, Antonio Torralba. The IEEE Conference on Computer Vision and Pattern Recognition (CVPR), 2016, pp. 2921-2929.
34. Coleman K, Baak J P, van Diest P J, Mullaney J. Prognostic value of morphometric features and the callender classification in uveal melanomas. Ophthalmology. 1996; 103(10):1634-1641.
35. Gamel J W, McCurdy J B, McLean I W. A comparison of prognostic covariates for uveal melanoma. Invest Ophthalmol Vis Sci. 1992; 33(6):1919-1922.
36. McLean I W, Sibug M E, Becker R L, McCurdy J B. Uveal melanoma: the importance of large nucleoli in predicting patient outcome—an automated image analysis study. Cancer. 1997; 79(5):982-988.
37. Correa Z M, Augsburger J J. Independent Prognostic Significance of Gene Expression Profile Class and Largest Basal Diameter of Posterior Uveal Melanomas. Am J Ophthalmol. 2016; 162:20-27 e21.
38. McLean M J, Foster W D, Zimmerman L E. Prognostic factors in small malignant melanomas of choroid and ciliary body. Arch Ophthalmol. 1977; 95(1):48-58.
39. Lane A M, Kim I K, Gragoudas E S. Survival Rates in Patients After Treatment for Metastasis From Uveal Melanoma. JAMA Ophthalmol. 2018; 136(9):981-986.
40. Correa Z M, Augsburger J J. Sufficiency of FNAB aspirates of posterior uveal melanoma for cytologic versus GEP classification in 159 patients, and relative prognostic significance of these classifications. Graefes Arch Clin Exp Ophthalmol. 2014; 252(1):131-135.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting an ophthalmologic genetic disease in a subject at least partially using a computer, the method comprising matching, by the computer, one or more properties of one or more images of one or more ocular tissues or portions thereof from the subject with one or more properties of at least one ocular pathology model that is trained on a plurality of reference images of ocular tissues or portions thereof from reference subjects, which properties of the ocular pathology model are indicative of the ophthalmologic genetic disease, thereby detecting the ophthalmologic genetic disease in the subject.

2. A method of classifying uveal melanoma tissues or portions thereof in a subject at least partially using a computer, the method comprising matching, by the computer, one or more properties of one or more images of one or more uveal melanoma tissues or portions thereof from the subject with one or more properties of at least one uveal melanoma model that is trained on a plurality of reference images of uveal melanoma tissues or portions thereof from reference subjects, which properties of the uveal melanoma model are indicative of a survival outcome prediction of the uveal melanoma, thereby classifying the uveal melanoma cells in the subject.

3. The method of claim 1, comprising:
dividing, by the computer, the reference images of the ocular tissues or portions thereof from the reference subjects into at least two tiles to generate tile sets, which ocular tissues or portions thereof comprise a given ocular pathology;
retaining, by the computer, tiles in the tile sets that comprise images of the ocular tissues or portions thereof that comprise the given ocular pathology to generate retained tile sets; and,
inputting, by the computer, the retained tile sets into a neural network comprising a classification layer that outputs survival outcome predictions for the given ocular pathology to train the neural network, thereby producing the ocular pathology model.

4. (canceled)

5. The ocular pathology model produced by the method of claim 3.

6. The method of claim 1, wherein the ophthalmologic genetic disease comprises cancer.

7. The method of claim 3, wherein the classification layer comprises a binary classification layer that classifies uveal melanoma samples as gene expression profile (GEP) class 1 or GEP class 2.

8. The method of claim 1, comprising obtaining the ocular tissues or portions thereof from the subject.

9. The method of claim 1, wherein the properties comprise one or more patterns.

10. The method of claim 1, further comprising administering one or more therapies to the subject to treat the ophthalmologic genetic disease.

11. The method of claim 1, further comprising repeating the method at one or more later time points to monitor progression of the ophthalmologic genetic disease in the subject.

12. The method of claim 3, wherein the ocular pathology model comprises one or more selected therapies indexed to the ocular pathology of the subject.

13. The method of claim 1, comprising capturing the images of the ocular tissues or portions thereof from the subject with a camera.

14. The method of claim 13, wherein the camera is operably connected to a database comprising an electronic medical record of the subject and wherein the method further comprises retrieving data from the electronic medical record and/or populating the electronic medical record with at least one of the images and/or information related thereto.

15. The method of claim 13, wherein the camera is wirelessly connected, or connectable, to the electronic medical record of the subject.

16. The method of claim 14, wherein the camera and/or the database is wirelessly connected, or connectable, to one or more communication devices of one or more remote users and wherein the remote users view at least one of the images of the ocular tissues or portions thereof of the subject and/or the electronic medical record of the subject using the communication devices.

17. The method of claim 16, wherein the communication devices comprise one or more mobile applications that operably interface with the camera and/or the database.

18. The method of claim 16, wherein the users input one or more entries into the electronic medical record of the subject in view of the detected ocular pathology of the subject using the communication devices, and/or wherein the users order one or more therapies and/or additional analyses of the subject in view of the detected ocular pathology of the subject using the communication devices.

19. (canceled)

20. The method of claim 14, wherein a system that comprises the database automatically orders one or more therapies and/or additional analyses of the subject in view of the detected ocular pathology of the subject when the users input the entries into the electronic medical record of the subject.

21. The method of claim 1, wherein the tissues or portions thereof comprise cells, organelles, and/or biomolecules.

22. The method of claim 2, wherein the survival outcome prediction comprises a gene expression profile (GEP) class.

23. A system, comprising:
at least one camera that is configured to capture one or more images of ocular tissues or portions thereof from a subject;
at least one controller that is operably connected, or connectable, at least to the camera, wherein the controller comprises, or is capable of accessing, computer readable media comprising non-transitory computer executable instructions which, when executed by at least one electronic processor, perform at least:
capturing the images of the ocular tissues or portions thereof from the subject with the camera to generate captured images; and,
matching one or more properties of the captured images with one or more properties of at least one ocular pathology model that is trained on a plurality of reference images of ocular tissues or portions thereof of reference subjects, which properties of the ocular pathology model are indicative of at least one ocular pathology.

24. (canceled)
